(19) United States
(12) Patent Application Publication
(10) Pub. No.: US 2020/0054492 A1
(43) Pub. Date: Feb. 20, 2020

(54) LIQUID PROPHYLACTIC ANKLE BRACE

(71) Applicant: UNIVERSITY OF FLORIDA RESEARCH FOUNDATION, INC., Gainesville, FL (US)

(72) Inventors: Ghatu SUBHASH, Gainesville, FL (US); Goeto DANTES, Gainesville, FL (US)

(21) Appl. No.: 16/345,925

(22) PCT Filed: Oct. 25, 2017
(86) PCT No.: PCT/US2017/058280

§ 371 Date: Apr. 29, 2019

(51) Int. Cl. A61F 13/06

(52) U.S. Cl. CPC A61F 13/066

(57) ABSTRACT

A protective ankle brace has at least one pocket that houses at least one pouch, and at least one strap, and covers both sides of an ankle of a foot placed within the ankle brace. The pouch contain a dilatant fluid that is flexible to allow normal motion for walking and running but on a rapid movement or one with a high force, the dilatant resists movement. The at least one strap covers the one or two pockets to retain the pouch or pouches. The strap restricts the maximum eversion and inversion of the ankle brace when a force is applied and further protects the ankle within the ankle brace. The strap can be a bladder containing a dilatant fluid.

### CROSS-REFERENCE TO A RELATED APPLICATION

This application claims the benefit of U.S. Provisional Application Ser. No. 62/414,954, filed Oct. 31, 2016, the disclosure of which is hereby incorporated by reference in its entirety, including all figures, tables and drawings.

### BACKGROUND OF INVENTION

Incidence of lateral ankle sprains stands as a plague for both athletes and non-athletes alike. As one of the most common injuries incurred in recreational, military-training and competitive athletics, these injuries significantly impact lives in tennis of cost, well-being, athletic participation, and activities of daily living. Incidence rate as high as an ankle sprain per 1,163 playing hours for male soccer players with a previous history of ankle sprain and an ankle sprain per 2,174 playing hours for previously uninjured ankles has been determined. Among competitive adult male soccer players, a survey reported 25% ankle sprains among unbraced athletes with a previous history of ankle sprain injury and even higher rates among women soccer players every season. Ankle injuries account for 30 to 60% of all parachuting injuries. These high rates of ankle injury are found in most major sports involving jumping, landing, and cutting maneuvers. Certain occupations and normal tasks of daily living also regularly result in ankle injuries.

Current prophylactic measures have significantly reduced the occurrence of ankle sprains. However, although dependent on a patient's level of compliance, recent epidemiological studies show that ankle sprains still account for 14% of all attendances at an accident and emergency department. Factors found to influence compliance with a prophylactic bracing include comfort, weight, ease of application, and perceived impact on performance. The current market offers a wide variety of braces that show mixed reviews of efficacy and affect on performance.

The challenge for prophylactic bracing is to achieve balance between free movement of the ankle and protection, as both factors are extremely important for optimal performance over time. Providing more protection of the ankle often results with a sacrifice of the range of motion or to comfort. Recently, an attempt in designing an intelligent sprain free sport shoe for preventing ankle sprain injury by using an insert with complicated electronics, and probably a high cost, provides unnecessary protection in 14.3% of the trials and fails to provide protection in 3% of the trials.

The anterior talofibular ligament (ATFL) requires the lowest maximal load to fail, which defines a threshold for lateral ligamentous sprain and, therefore, sets the minimal required resistance to provide some reliable protection against ankle sprains. Unfortunately, currently there are very limited and conflicting statistics indicating exact kinetic measures delineating sprains. Mainly, because of medical ethics, studies haves been limited to examination of post-mortem cadavers or animal models that fail to factor surrounding tissue and musculature. St. Pierre et al., “The Tensile Strength of the Anterior Talofibular Ligament” Foot & Ankle 4(2) 83, 1983, discloses tests of 36 human ATFL specimens for tensile strength at a relatively slow displacement rate (12.5cm/min) and sets an average strength at 206 N, with a range of 58 to 556 N.

Siegler et al., “The Mechanical Characteristics of the Collateral Ligaments of the Human Ankle Joint” Foot & Ankle 8(5):234, 1988, in vitro tensile tested 120 ligaments obtained from 20 fresh lower limbs at a low stretch rate of 0.32 cm/min such that any viscous effects could be neglected. This article examined the yield point, which is that extension where some ligament fibers have failed but most of the support structure remains intact, the ultimate point, which is the extension at which major ligament failure occurs due to ligament tearing or bone avulsion, and the linear loading region. This study found ATFL, the shortest ligament at about 1.8 cm exhibited the lowest yield force (222.0 N) and ultimate load (231.0 N) of the lateral collateral ligaments.

Attarian et al. “A Biomechanical Study of Human Lateral Ankle Ligaments and Autogenous Reconstruction Grafts” Am. J. Sports Med. 13(6):377, 1985 conducted cyclic loading at physiologic deflections, several load-deflection tests at varying velocities, and an extremely rapid (80 to 100 cm/sec) load to failure test. This study found maximum load of the ATFL for a 12 specimen sample of 138.9±23.5 Newtons. Chu et al. “Differentiation of Ankle Sprain Motion and Common Sporting Motion by Ankle Inversion Velocity” J. Biomechanics 43(10):2035, 2010, investigated ankle inversion and inversion velocity for various common motions in sports and simulated sprain motion to provide a threshold for ankle sprain risk identification. Little difference in the inversion angle was found but showed that the inversion velocity of simulated sprain motion is much greater than the common sporting motion. Their research concluded a safe threshold of 300 deg/sec. Fong et al. “Kinematics Analysis of Ankle Inversion Ligamentous Sprain Injuries in Sports Five Cases From Televised Tennis Competitions” Am. J. Sports Med. 2012 40(11):2627, 2012, conducted image-matching motion analysis of five sets of videos showing ankle sprain injuries in televised tennis competition with peak inversion velocity of injury ranging from 509 to 1488 deg/sec. This was consistent with a safety threshold of 300 deg/sec as an angular velocity that allows common sport motions, yet can be reasonably believed to be below those velocities that cause ankle sprains.

Therefore, a device that can permit up to 300 deg/sec of motion but can resist a force of about 200 N could significantly reduce the incidence of ankle sprain.

### BRIEF DESCRIPTION OF DRAWINGS

FIG. 1 is a drawing of an ankle brace, according to an embodiment of the invention.

### DETAILED DISCLOSURE

In an embodiment of the invention, a prophylactic brace that effectively protects the ankle from injury while providing acceptable levels of comfort, weight, ease of wearing, and athletic performance includes a non-Newtonian shear thickening fluid filled bladder that is secured and reinforced at a maximal allowable displacement in a brace device. The device provides unobstructed movement at normal ranges, speeds, and degrees of ankle motion yet provides a rapidly responding counter to potentially damaging displacements and rates of shear. By titrating the fluid's response to a wearer's threshold for injury this brace does not sacrifice any motion to provide protection. By use of the non-Newtonian fluid, small amounts of the fluid provide efficacious protection at a cost of little weight, size, and restriction to necessary motion that could adversely impact performance. This would mean that the brace could potentially be used in various sports and activities, with little impact on normal ankle kinematics.

In an embodiment of the invention, the ankle brace provides a method to prevent medial and lateral ankle sprains. This ankle brace allows the ankle to move freely at velocities that do not impede athletic performance, but affords protection of the ankle when a high rotational and/or shear stress is applied to the ankle. It is an ankle strap consisting of two fluid gel, Newtonian or non-Newtonian liquid, or any other type of fluid filled pouches on both sides of an ankle to provide additional support during a physical activity. The pouches are placed in pockets that are can be fixed on either side of a commercially available ankle bracelet at appropriate positions. Additional one or more straps on the brace are situated over the pouches and secure them tightly against the ankle, and provide additional support. When an ankle twists during a physical activity, the non-Newtonian liquid in the tightly secured pouches will act against the rotation of the ankle to prevent injury. Additionally, the straps limit the degree of deflection permitted of the ankle, such that deflection or rotation of the ankle is limited to the degree permitted by the strap.

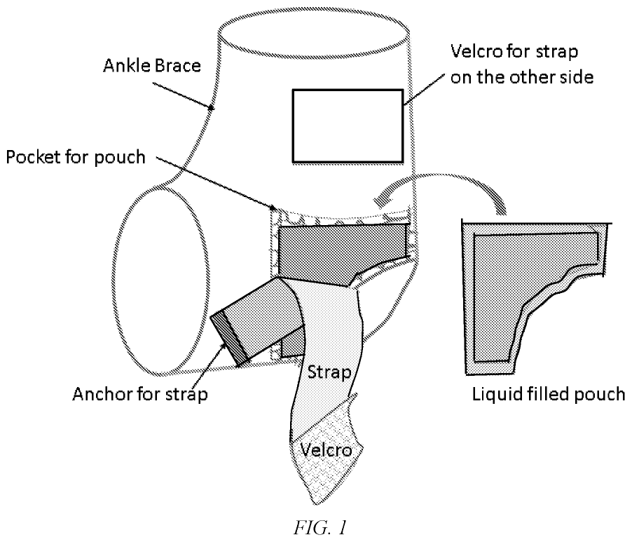

The prophylactic brace, according to an embodiment of the invention, has two or more pockets situated on the inner and outer portion of the ankle, particularly situated to cover the area of the lateral collateral ligament, the anterior talofibular ligament, the posterior talofilular ligament and the calconofibular ligament. These ligaments are those which tear under the lowest stress and/or are situated such that twisting and inversion occurs. The pockets receive pouches that comprises a Non-Newtonian liquid, which is a dilatant that shear thickens at a rapid rate when a rapid movement is applied, as when an inversion of the ankle under a leg occurs. Additionally, the dilatant can guard against impact, when the ankle brace is used in an activity where an impact is possible, for example in a military use or a contact sport. The pouches can be of a C-shape that extends over the ankle and downward towards the arch of the foot and backward towards the Achilles tendon, as shown in FIG. 1. In other embodiments of the invention the pouches and the pockets can be of a T-shape where an additional portion extends in from of the ankle towards the front of the leg. The two pockets and pouches that are situated on the inner and outer sides of the foot, can be connected in front or behind the ankle, for example, where the backward portions, as shown in FIG. 1, are attached or continuous, being connected or continuous behind the ankle, or the two T-shaped pockets and pouches can be connected or can be continuous over the front of the ankle. The thickness of the pockets with the pouches inserted can be about 2 mm to about 10 mm or more. Other dimensions can vary significantly for shape of the pouches and pockets and for the size of the ankle to be housed therein, as can be appreciated by one of ordinary skill in the art.

In an embodiment of the invention, at least two straps proceed from fixed securing positions on the brace on the foot forward and below the ankle; are extended over the pocket and pouch and the ankle; are extended over the Achilles tendon behind the ankle; and are secured, as shown in FIG. 1, on opposing sides of the ankle on the leg above the ankle. Alternatively, the two straps proceed from fixed securing positions on the leg above the ankle; are extended over the pocket and pouch and the ankle; are extended over the Achilles tendon behind the ankle; and are secured on the brace on the foot forward and below the ankle. In another embodiment of the invention, a single strap is employed, where the strap originates from a fixed securing position on the brace on the foot forward and below the ankle; is extended over a pocket and pouch and the ankle on one side of the brace; is extended over the Achilles tendon behind the ankle; extended around the front of the leg immediately above the ankle and is secured on the brace on the foot forward and below the ankle on the opposite side of the origin. The strap or straps do not significantly affect the plantarflexion and dorsiflexion of the ankle, but resist and/or limit the ultimate eversion and inversion of the foot while providing sufficient flexibility for small eversions and inversions, as would occur during normal walking and running.

The dilatant non-Newtonian fluid included within a pouch can be, but are not limited to, corn starch dispersed in water, silica nanoparticles dispersed in polyethylene glycol, and polyborodimethylsiloxane. The pouch can be a flexible plastic or a rubber of any sort, including polyethylene, polypropylene, polyisoprene, polybutadiene, polysiloxane, polyurethane, or any other plastic or rubber. The strap can comprise a composite of a rubber and aligned non-elastic fiber, to define a limit to the extension but allow flexibility in the perpendicular planes to facilitate comfort and functionality of attachment. The straps can be any woven or non-woven fabric of, for example, but not limited to, polyamides, polyolefins, polyesters, polyaramides, or any other polymer that can flex but does not elongate significantly when under stress. The straps can be a fiber filled rubber or plastic body, where the fibers can be of a sufficient length to sufficiently reinforce as an ensemble, including the entire length of the strap. The straps or composite fillers can be woven or otherwise connected metal links or fiber. The fasteners can be of a hook and loop (Velcro™), Dual Lock™, buttons, Xolok™, or any other method of fixing the straps reliably with sufficient resistance to breakage or opening. In an embodiment of the invention, the strap can be a rubber bladder that is filled with a dilatant fluid that is the same or different than the dilatant fluid in the pouches.

The ankle brace is of sufficient thinness that it can be fit within a shoe, although, in embodiments of the invention, the shoe can have a structure that accepts the addition of the pouches. The shoe may have additional cavities to accommodate the pouches and straps and can include additional fasteners to reinforce and fix the position of the ankle brace within the shoe.

In another embodiment of the invention, the ankle brace can be a portion of a shoe, wherein the shoe includes the at least one pocket for receiving the at least one pouch. In this manner the shoe and brace necessarily fit and the straps are included for resisting a maximum eversion and inversion possible while having the proximal attachment of the strap being within or on the outside of the shoe. In this manner, the movement of the shoe is coupled with the movement of the brace.

All publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A protective ankle brace comprising: at least one pocket; at least one pouch; and at least one strap, wherein the at least one pocket houses the at least one pouch and situated to support and cover both sides of an ankle of a foot placed within the ankle brace, wherein the pouch contains a dilatant fluid, wherein the at least one strap covers the at least one pocket and retains the at least one pouch in the at least one pocket, and wherein the at least one strap restricts the maximum eversions and inversions of the ankle brace and the ankle within the ankle brace.

2. The protective ankle brace according to claim 1, wherein the at least one pocket comprises and inner pocket and an outer pocket and the at least one pouch comprises an inner pouch and an outer pouch, as related to the ankle within the ankle brace.

3. The protective ankle brace according to claim 2, wherein the inner pocket, the outer pocket, the inner pouch and the outer pouch are of a C-shape.

4. The protective ankle brace according to claim 2, wherein the inner pocket, the outer pocket, the inner pouch and the outer pouch are of a T-shape.

5. The protective ankle brace according to claim 1, wherein the at least one pocket is a single pocket and the pouch is a single pouch, wherein the single pocket and the single pouch are continuous or connected to extend over the front of the ankle or over the back of the ankle having an inner portion and an outer portion relative to the ankle within the ankle brace.

6. The protective ankle brace according to claim 1, wherein the dilatant fluid is selected from: corn starch dispersed in water, silica nanoparticles dispersed in polyethylene glycol, and polyborodimethylsiloxane

7. The protective ankle brace according to claim 1, wherein the strap is a rubber, plastic, composite or metal and is a woven or non-woven body.

8. The protective ankle brace according to claim 1, wherein the strap comprises a rubber bladder that is filled with a dilatant fluid.

9. The protective ankle brace according to claim 1, wherein the ankle bracelet is a portion of a shoe.
